(19) United States
(12) Patent Application Publication
(10) Pub. No.: US 2003/0224995 A1
(43) Pub. Date: Dec. 4, 2003

(54) Treatment of burns

(76) Inventors: Nisar Ahmed Khan, Rotterdam (NL); Gert Wensvoort, Koekange (NL); Robbert Benner, Barendrecht (NL)

Correspondence Address: TRASK BRITT, P.O. BOX 2550, SALT LAKE CITY, UT, 84110, US

(21) Appl. No.: 10/409,694

(22) Filed: Apr. 8, 2003

Related U.S. Application Data
(63) Continuation-in-part of application No. 10/028,075, filed on Dec. 21, 2001.

(51) Int. Cl. A61K 38/17

(52) U.S. Cl. 514/12

(57) ABSTRACT

The invention relates to the treatment of burn injuries. The invention provides a method for modulating a burn injury in a subject including providing the subject with a gene-regulatory peptide or functional analogue thereof. Furthermore, the invention provides use of an NF-κB down-regulating peptide or functional analogue thereof for the production of a pharmaceutical composition for the treatment of burn injury of a subject.

### CROSS-REFERENCE TO RELATED APPLICATION

This application is a continuation-in-part of U.S. application Ser. No. 10/028,075, filed Dec. 21, 2001, pending, the contents of the entirety of which is incorporated by this reference.

### TECHNICAL FIELD

The current invention relates to the body's innate way of modulation of important physiological processes and builds on insights reported in PCT International Publications WO99/59617 and WO 01/72831 and PCT International Application PCT/NL02/00639, the contents of the entirety of all of which are incorporated by this reference.

### BACKGROUND

In the aforementioned patent applications, small gene-regulatory peptides are described that are present naturally in pregnant women and are derived from proteolytic breakdown of placental gonadotropins such as human chorionic gonadotropin (hCG) produced during pregnancy. These peptides (in their active state often only at about 4 to 6 amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except to serve as antigen for the immune system), it now emerges that the body in fact routinely utilizes the normal process of proteolytic breakdown of the proteins it produces to generate important gene-regulatory compounds, short peptides that control the expression of the body's own genes. Apparently, the body uses a gene-control system ruled by small broken down products of the exact proteins that are encoded by its own genes.

It is known that during pregnancy the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

It was shown that certain short breakdown products of hCG (i.e., short peptides which can easily be synthesized, if needed modified, and used as pharmaceutical composition) exert a major regulatory activity on pro- or anti-inflammatory cytokine cascades that are governed by a family of crucial transcription factors, the NFκB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFκB-mediated expression of cytokine genes, thereby keeping the immunological responses in the placenta at bay.

### BRIEF SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFκB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFκB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### DETAILED DESCRIPTION OF THE INVENTION

The invention in particular relates to the treatment of burn injuries.

Bum injuries are among the worst traumas which can happen to man. The larger a burn injury, the more severe the consequences and the higher the chance of an adverse outcome or even death. In The Netherlands each year 40,000 people visit a general practitioner for treatment of a burn wound and 1600 people require in-hospital care primarily for burns.

Approximately 80% of the burn accidents happen in or around the house; mainly in the kitchen. Scalds, usually due to hot water, are the most common cause of burns. Water at 60° C. will create a deep dermal or full-thickness burn in three seconds, and at 70° C. the same burn will occur in one second. The temperature of freshly brewed coffee from a percolator is generally about 80° C., which is hot enough to cause a full-thickness burn in less than one second. Children are particularly at high risk to burns. Hot beverages, particularly coffee and tea, are the predominant cause of scald burns in children. One study showed that 81% of the burn injuries in children under the age of 5 were due to scalds. Cooking oil, when hot enough to use for cooking, may be in the range of 150-180° C. and can consequently cause very severe burns. Bums are estimated to affect >1.4 million people in the United States annually. Of this number, 54.000 patients require hospital admission, 16.000 of whom have injuries of such significance that care is best undertaken in a burn center. House and structure fires are responsible for >70% of the yearly 5.400 burn-related deaths, of which three fourths result from smoke inhalation or asphyxiation and one fourth are due to burns. However, these fires are responsible for only 4% of burn admissions. Injuries due to contact with flame or ignition of clothing are the most common cause of burn in adults, whereas scald burns are most common in children. The majority of patients sustain burns limited severity and extent (>80% of burns involve <20% of the body surface) that they can be treated on an outpatient basis. There are 250 to 275 patients per million population per year who require hospital admission owing to the extent of their burns or to other complicating factors. Approximately one third of patients who require in-hospital care have a major burn injury- as defined by the American Bum Association on the basis of burn size, causative agent, pre-existing disease, and associated injuries- and should be treated in a tertiary care burn center. Other causes of burns are fire, electricity, chemical substances and even sunshine. In The Netherlands, around 200 people die of burn incidents each year, mostly at the place of the accident. The case fatality rate of scald injury is low; instead most deaths occur in residential fires, commonly caused by careless smoking, by arson or by defective or inappropriately used heating devices. The skin consists of two morphologically different layers that are derived from two different germ layers. The more superficial layer, the epidermis, is a specialized epithelial tissue derived from surface ectoderm. The deeper and thicker layer, the dermis, is composed of vascular dense connective tissue derived from mesenchyme. In recent years the concept of the epidermis has gradually been changing from that of an innocent bystander, that, in its strictest sense, protects the body from the loss of fluids and electrolytes, and the penetration of harmful substances, into that of an active participant in several important processes. The dermis is situated between the epidermis and the subcutaneous fat. It supports the epidermis structurally and nutritionally. Its thickness varies, being greatest in the palms and soles and the least in the eyelids. With aging the dermis becomes thinner and loses elasticity. The dermis interdigitates with the epidermis, so that the upward projections of the dermis, the dermal papillae, interlock with downward ridges of the epidermis, the rete ridges. Like all connective tissue, the dermis has three components: cells, fibers and amorphous ground substance. The bulk of the dermis consists of a network of fibers, principally collagen, but also reticulin and elastin, packed in bundles. Those in the papillary dermis being finer than those in the deeper, reticular dermis. The amorphous ground substance of the dermis consists largely of two glycosaminoglycans: hyaluronic acid and dermatan sulfate, with smaller amounts of heparan and chondroitin sulfate. The function of the ground substance is that it binds water, in order to allow nutrients, hormones and waste products to pass through the dermis. It also is a lubricant between the collagen and elastic fiber network during skin movement and it provides bulk, allowing the dermis to act as a shock absorber. The dermis also contains muscles, both smooth and striated, and vessels. Blood vessels are not only necessary for feeding, but also for regulation of the body temperature. Besides that, blood vessels play a role in allowing transendothelial migration of immune cells, by expressing adhesion molecules that bind to receptor molecules on the immune cells. This transmigration process allows immune cells into the tissue to do their surveillance work. Lymphatic vessels, beginning as blind-ended capillaries in the dermal papillae, pass to either the superficial lymphatic plexus in the papillary dermis, or to the deeper horizontal plexuses. They play a role in water homeostasis of the dermal tissue and also in the recirculation of immune cells.

Thermal energy is a manifestation of random molecular kinetic energy. This energy is easily transferred from high energy molecules to those with a lower energy status during contact, for example in living tissues. Both the temperature and the time period for which this temperature is sustained determine the degree of damage to a cell. At temperatures between 40 and 44° C., various enzyme systems begin to malfunction, and early denaturation of protein occurs. Cellular functions become impaired, one of which is the membrane Na⁺ pump. This results in a high intracellular Na⁺ concentration and concomitant swelling of the cell. As the temperature increases, damage accumulation outruns the cell's inherent repair mechanisms and leads to eventual necrosis. The production of oxygen free radicals is part of this damage process. These highly reactive molecules are capable of promoting further cell membrane abnormalities, leading to cell death. If the heat source is suddenly withdrawn, damage accumulation will continue until the cooling process brings cells back down to a normal temperature range. Cooling determines the difference between cell survival and cell death.

As the temperature increases, protein coagulation takes place, which causes destruction of the protein architecture. New aberrant bonds are formed, creating macromolecules not similar to the original structures. The cell necrosis is complete, usually beginning at the skin surface, where the heat energy was absorbed most directly, extending downward. This zone is called the zone of coagulation. The zone of stasis lies deeper and peripheral to the zone of coagulation. In this zone the damage is less and most cells are initially viable. However, the blood flow becomes progressively impaired and finally stops. This development of ischaemia results in necrosis of the already affected cells. Peripheral to this zone lays the zone of hyperemia, which is characterized by minimal cellular injury and prominent vasodilatation with increased blood flow, due to vasoactive mediators that were produced as part of the inflammatory response. Complete cellular recovery usually happens from this zone up only when capillaries will grow back upward. Bums can be divided into different categories, based on the depth level of the tissue damage. First degree burn injury involves damage only to the epidermis and is rarely clinically significant other than being painful. The involved area is initially erythematous due to vasodilitation. Eventually desquamation happens, but this is followed by complete scarless healing within 7 days. Second degree burns are partial-thickness by definition and are further categorized into superficial and deep. In superficial injuries, the epidermis is destroyed as well as varying superficial portions of the dermis. These lesions are usually painful. Blistering is often present. Healing generally occurs rapidly and completely through migration to the surface of epithelial stem cells which survive in deeper portions of the hair follicles as well as the sweat and sebaceous glands. Relatively little scarring occurs in a superficial injury, due to the limited inflammatory phase, which is cut short by wound closure (re-epithelialization) occurring within 2 weeks. In deep partial-thickness wounds most of the dermis is destroyed and only in the deepest parts of the hair follicles, sweat and sebaceous glands few epithelial cells remain. As the epithelial cells have to migrate from the depth, and due to the loss of stem cells, re-epithelialization is greatly retarded in these wounds. Heat kills the superficial nerve endings, so the wound is relatively insensitive. As the deeply situated pressure receptors may survive, pressure sensation can still be present. Blistering is usually absent due to the thicker adherent overlying eschar which prevents the lifting by the edema. Due to the long period wound closure, the inflammatory phase is prolonged, which gives rise to extensive collagen deposition and consequently abundant scar formation. In third degree or full-thickness burns necrosis of the entire thickness of the skin occurs. As there are no epithelial appendages left, healing can only occur by re-epithelialization from the wound edges, or, in case of small wounds, by contraction of the wound edges. So third degree wounds are routinely treated with excision and skin grafting, serving as a source of new stem cells. As no nerve endings are left, this type of wound is insensitive. Infection, the risk of which is proportional to the extent of injury, continues to be the predominant determinant of outcome in thermally injured patients despite improvements in overall care in general and wound care in particular. In particular, as a manifestation of the systemic immunosuppressive effects of burn injury, infection at other sites, predominantly in the lungs, remains the most typical cause of morbidity and death in these severely injured patients. Bum patients with or without inhalation injury commonly exhibit a clinical picture produced by systemic inflammation. The phrase “systemic” inflammatory response syndrome (“SIRS”) has been introduced to designate the signs and symptoms of patients suffering from such a condition. SIRS has a continuum of severity ranging from the presence of tachycardia, tachypnea, fever and leukocytosis, to refractory hypotension and, in its most severe form, shock and multiple organ system dysfunction. In thermally injured patients, the most common cause of SIRS is the burn itself. Sepsis, SIRS with the presence of infection or bacteremia, is also a common occurrence. Pathological alterations of metabolic, cardiovascular, gastrointestinal, and coagulation systems occur as a result of the hyperactive immune system. Paradoxically, a state of immunosuppression often follows or co-exists with SIRS. The counter antiinflammatory response syndrome (CARS) appears to be an adaptive mechanism designed to limit the injurious effects of systemic inflammation. However, this response may also render the host more susceptible to systemic infection due to impaired antimicrobial immunity. Both cellular and humoral mechanisms are involved in these disease processes and have been extensively studied in various burn and sepsis models. The phrase systemic inflammatory response syndrome (SIRS) was recommended by the American College of Chest Physicians/Society for Critical Care Medicine (ACCP/SCCM) consensus conference in 1992 to describe a systemic inflammatory process, independent of its cause. The proposal was based on clinical and experimental results indicating that a variety of conditions, both infectious and noninfectious (i.e., burns, ischemia-reperfusion injury, multiple trauma, pancreatitis), induce a similar host response. Two or more of the following conditions must be fulfilled for the diagnosis of SIRS to be made:

Body temperature >38° C. or <36° C.;

Heart rate >90 beats/min.;

Respiratory rate >20/min or Paco₂<32 mmHg;

Leukocyte count >12.000/1μ, <4000 μL, or >10% immature (band) forms

All of these pathophysiologic changes must occur as an acute alteration from baseline in the absence of other known causes for them such as chemotherapy-induced neutropenia and leukopenia.

The control of invasive burn wound infection through the use of effective topical chemotherapy, prompt surgical excision, and timely closure of the burn wound has resulted in unsurpassed survival rates. Even so, infection remains the most common cause of death in these severely injured patients.

Changes in wound care over the past thirty years, including the use of effective topical antimicrobial chemotherapy and excision of the burned tissue to achieve timely closure of the burn wound, have significantly reduced the occurrence of invasive burn wound infection and its related morbidity and mortality. Regular collection of cultures from patients permits early identification of the causative pathogens of those infections that do arise. Moreover, infection control procedures, including strict enforcement of patient and staff hygiene and use of patient isolation methods, have been effective in controlling the spread of resistant organisms and eliminating them from the burn centre. These advances and the improvements in the general care of critically ill burn patients have resulted in markedly improved survival rates.

The invention provides a method for modulating or treating a burn injury in a subject believed to be in need thereof comprising providing the subject with a signaling molecule comprising a gene-regulatory peptide or functional analogue thereof wherein the signaling molecule is administered in an amount sufficient to modulate the burn injury. The signal molecule is preferably a short peptide, preferably of at most 30 amino acids long, or a functional analogue or derivative thereof. In a much preferred embodiment, the peptide is an oligopeptide of from about 3 to about 15 amino acids long, preferably 4 to 12, more preferably 4 to 9, most preferably 4 to 6 amino acids long, or a functional analogue or derivative thereof. Of course, such a signaling molecule can be longer, for example by extending it (N- and/or C-terminally), with more amino acids or other side groups, which can for example be (enzymatically) cleaved off when the molecule enters the place of final destination. In particular a method is provided wherein the signaling molecule modulates translocation and/or activity of a gene transcription factor. It is particularly useful when the gene transcription factor comprises an NF-κB/Rel protein or an AP-1 protein. Burn injuries generally induce increased expression of inflammatory cytokines due to activation of NF-κB and AP-1, and in a preferred embodiment the invention provides a method wherein translocation and/or activity of the NF-κB/Rel protein is inhibited. In one embodiment, the peptide is selected from the group of peptides LQG, AQG, LQGV (SEQ ID NO: 1 of the hereby incorporated accompanying SEQUENCE LISTING), AQGV (SEQ ID NO: 2), LQGA (SEQ ID NO: 3), VLPALP (SEQ ID NO: 4), ALPALP (SEQ ID NO: 5), VAPALP (SEQ ID NO: 6), ALPALPQ (SEQ ID NO: 7), VLPAAPQ (SEQ ID NO: 8), VLPALAQ (SEQ ID NO: 9), LAGV (SEQ ID NO: 10), VLAALP (SEQ ID NO: 11), VLPALA (SEQ ID NO: 12), VLPALPQ (SEQ ID NO: 13), VLAALPQ (SEQ ID NO: 14), VLPALPA (SEQ ID NO: 15), GVLPALP (SEQ ID NO: 16), LQGVLPALPQVVC (SEQ ID NO: 17), LPGCPRGVNPVVS (SEQ ID NO: 18), LPGC (SEQ ID NO: 19), MTRV (SEQ ID NO: 20), MTR, VVC. Burn injury induces increased expression of inflammatory cytokines due to activation of NF-κB and AP-1. Inflammatory cytokines can be expressed by epithelium, perivascular cells and adherent or transmigrating leukocytes, inducing numerous pro-inflammatory and procoagulant effects. Together these effects predispose to inflammation, thrombosis and hemorrhage. Of clinical and medical interest and value, the present invention provides the opportunity to selectively control NFκB-dependent gene expression in tissues and organs in a living subject, preferably in a primate, allowing upregulating essentially anti-inflammatory responses such as IL-10, and downregulating essentially pro-inflammatory responses such as mediated by TNF-α, nitric oxide (NO), IL-5, IL-1β.

The invention is further explained with the aid of the following illustrative examples.

### EXAMPLES

The invention thus provides use of a NFκB regulating peptide or derivative thereof for the production of a pharmaceutical composition for the treatment of a burn injury, preferably in a primate, and provides a method of treatment of a burn injury, notably in a primate. It is preferred when the treatment comprises administering to the subject a pharmaceutical composition comprising an NFκB down-regulating peptide or functional analogue thereof. Examples of useful NFκB down-regulating peptides are VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VVC, MTR and circular LQGVLPALPQVVC (SEQ ID NO: 17). More down-regulating peptides and functional analogues can be found using the methods as provided herein. Most prominent among NFκB down-regulating peptides are VLPALPQVVC (SEQ ID NO: 21), LQGVLPALPQ (SEQ ID NO: 22), LQG, LQGV (SEQ ID NO: 1), and VLPALP (SEQ ID NO: 6). These are also capable of reducing production of NO by a cell. It is herein also provided to use a composition that comprises at least two oligopeptides or functional analogues thereof, each capable of down-regulating NFκB, and thereby reducing production of NO and/or TNF-α by a cell, in particular wherein the at least two oligopeptides are selected from the group LQGV (SEQ ID NO: 1), AQGV (SEQ ID NO: 2) and VLPALP (SEQ ID NO: 6), for the treatment of a burn injury, and, moreover to treat the systemic inflammatory response often seen in severe burn patients. The invention for this purpose provides use of a such signaling molecule comprising a NF-κB down-regulating peptide or functional analogue thereof for the production of a pharmaceutical composition for the treatment of a systemic inflammatory response syndrome occurring after a burn injury of a subject, in particular wherein translocation and/or activity of the NF-κB/Rel protein is inhibited, resulting in keeping the cascade of cytokine reactions that in general lead to SIRS at bay.

In general, when treating burns patients, two, often conflicting needs of the patient need be met. For one, the treatment of the affected, and locally seriously inflamed, skin deserves particular attention; on the other hand, the patient may also suffer from the consequences of a more systemic inflammatory response. Thermal injury initiates a deleterious pathophysiologic response in every organ system, with the extent and duration of organ dysfunction proportionate to the size of the burn. Direct cellular damage is manifested by coagulation necrosis, with the depth of tissue destruction determined by the duration of contact and the temperature to which the tissue is exposed. Following burn, the normal skin barrier to microbial penetration is lost, and the moist, protein-rich avascular eschar of the burn wound provides an excellent culture medium for microorganisms, that infect the burn injury. Although the body logically responds to these infections by eliciting a (local) inflammation, the invention provides use of a signaling molecule comprising a NF-κB down-regulating peptide or functional analogue thereof for the production of a pharmaceutical composition for the topical treatment of a burn wound in a subject, to actually counter the inflammation and prevent systemic responses and overly active scar tissue formation. The invention also provides a pharmaceutical composition comprising an NF-κB down-regulating peptide or functional analogue thereof and a bactericidal or bacteriostatic compound or a compound comprising silver. Wound management will vary according to the depth of the burn. The true depth of the burn will become more obvious with time and therefore the wound must be reassessed to ensure that wound management is appropriate. Systemic, and even topical, antibiotics are not to be used prophylactically, and are in general only appropriate when demonstrated infection is present, however, is in particular useful that translocation and/or activity of the NF-κB/Rel protein is inhibited to counter the local cytokine cascade leading to an inflammation by the inclusion of one or more of the NFκB down-regulating peptides or functional analogues thereof as identified herein, and at that time it is even more useful that the pharmaceutical composition for topical use is also provided with antibacterial compounds, preferably compounds that comprise silver, such as a antibacterial cream or ointment comprising micronized silver sulfadiazine and an NFκB down-regulating peptide. The invention thus provides a method to treat a burn injury of a subject wherein the subject is provided with a topical agent directed against a bacterial infection such as a bacteriostatic or bactericidal compound such as tetracycline or a sulfa compound wherein the topical agent also comprises a NFκB down-regulating peptide at a concentration of for example 1 to 1000 microg/g, preferably 50-300 microg/g. Typical other substances found in such a cream or ointment are 10 mg/gram of micronized silver sulfadiazine and a lege artis cream vehicle composed of white petrolatum, stearyl alcohol, isopropyl myristate, sorbitan monooleate, polyoxyl 40 stearate, propylene glycol, and water. Another anti-inflammatory and anti-infective cream for topical administration to burn wounds as herein provided comprises one or more of NFκB down-regulating peptides VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VVC, MTR at a concentration of for example 50-300 microgram/gram and contains per gram mafenide acetate equivalent to 85 mg of the base. The cream vehicle for example consists of cetyl alcohol, stearyl alcohol, cetyl esters wax, polyoxyl 40 stearate, polyoxyl 8 stearate, glycerin, and water, with methylparaben, propylparaben, sodium metabisulfite, and edetate disodium as preservatives may be added. While destruction of the mechanical barrier of the skin contributes to the increased susceptibility to infection, postburn alterations in immune function may also be of significant importance. Every component of the humoral and cellular limbs of the immune system appears to be affected after thermal injury; the magnitude and duration of dysfunction are proportional to the extent of injury. Wound healing is the consequence of a continuous sequence of signals and responses in which epithelial, vascular, hemopoietic and connective tissue cells come together outside their usual domains, interact, repair the damage and having done so turn back to their normal functions. The purpose of wound healing is to restore the functions of the skin, such as protection of the body against harmful environmental entities, prevention of entry of microorganisms and loss of plasma, the regulation of body temperature, the processing and interpretation of environmental information through the neurosensory system and a social-interactive function. Vertical cutaneous injuries, such as surgical incisions which have a minimal loss of tissue, will essentially heal through the formation of a blood clot, rapid epithelialization, and fibroblast proliferation. Progressive collagenization and increased strength, which reach normal levels within weeks, will complete the healing process and leave discrete scarring, in most cases. On the other hand, cutaneous wounds with a predominant horizontal loss of tissue, like burn injuries, exhibit a healing which proceeds through a series of complex, biological mechanisms according to the extent and level of the involved structures. In particular the destruction of the normal capillary system that is involved to the blood supply of the skin creates additional problems. A burn wound that suffers from decreased blood supply becomes ischemic, hypoxic, and highly edematous. For the various stages in the burn wound healing process use of a signaling molecule according to the invention for the preparation of a pharmaceutical composition for modulation of vascularization or angiogenesis in wound repair, in particular of burns, is herein provided. Not only may one treat topical and systemically with NFκB down-regulating peptides to find the best balance between a local inflammatory response while keeping systemic inflammation at bay; according to the invention one may also increase vasculogenesis by the topical application of modulatory peptides such LQG, VVC and MTRV (SEQ ID NO: 20), and in particular LQGV (SEQ ID NO: 1), which promote angiogenesis, especially in topical applications. Such angiogenesis-promoting compositions may be composed of 200-600 microg/ml of for example LQGV (SEQ ID NO: 1) in a gel vehicle that is for example composed of an oil-in-water emulsion base of glycerin, cetyl alcohol, stearic acid, glyceryl monostearate, mineral oil, polyoxyl 40 stearate and purified water. Of course, these can also be included in cream or ointment compositions as described above. In the absence of sufficient angiogenesis, burn wound healing follows a much slower course compared with the healing of other types of wounds. The wound healing response can be divided into three distinct, but overlapping phases: 1) hemostasis and inflammation; 2) dermal and epidermal proliferation; and 3) maturation and remodeling. The first response after disruption of tissue integrity, is to control the damage produced to the vascular system. A hemorrhage means immediate danger to the body, which reacts with prompt vasoconstriction, platelet aggregation and activation of the coagulation system. The initial response to deep burns involves a transient 5- to 10-minute period of intense vasoconstriction that aids in hemostasis. This is followed by active vasodilation that usually becomes most pronounced approximately 20 minutes after the injury and is accompanied by an increased capillary permeability. Histamine is believed to be a key chemical mediator responsible for the vasodilation and the danger in vascular permeability. Shortly after burning, platelet adhesion occurs at the site of the burn. Platelets function to initiate the formation of a clot that helps to achieve hemostasis. The contact between the extracellular matrix and platelets, as well as the presence of thrombin and fibronectin, results in the release of growth factors and vasoactive substances such as platelet-derived growth factor (PDGF), transforming growth factor-β (TGF-β), fibroblast growth factor (FGF), epidermal growth factor (EGF), bradykinin, prostaglandins, prostacyclins, thromboxane, histamine and serotonin. Platelet degranulation also initiates the complement cascade with the formation of C3a and C5a, which are potent anaphylatoxins promoting the release of histamine by basophils and mast cells. When angiogenesis is promoted in a method as provided herein these series of events are accompanied by improved blood supply from regenerating tissue which ultimately leads to less complicated wound healing. Also, granulocytes, in a rapid response to signalling by platelets and also through factors produced by the activation of the complement system, form the first line of defense against local bacterial contamination. In the absences of bacterial contamination, the granulocyte has been claimed to be non-essential to the wound healing process. Usually within 24-72 hours, the granulocytes are gradually replaced by monocytes that acquire the characteristics of tissue macrophages and become central coordinators of the inflammatory and repair process. Macrophages not only help to clean the wounded area of undesirable debris and bacteria, but they also promote the build up of the new connective tissue. Through growth factors and cytokines like TGF-β, PDGF and EGF, tumor necrosis factor-α (TNF-α), interleukin-1 (IL-1) and interferon-gamma (IFN-gamma), through enzymes like collagenase and arginase, and through prostaglandins, they regulate the matrix synthesis by affecting either fibroblast chemotaxis or proliferation, or collagen synthesis. Macrophages also play a role in mediating angiogenesis and in the recruitment and activation of other immune cells. By timely inclusion the use of NFκB down-regulating peptides in the treatment of burn injury, overly strong collagen matrix development can be modulated such that kyloid scars due to ridge formation are less well likely to develop. Furthermore, it has been demonstrated that activated T lymphocytes, following the influx of granulocytes and macrophages, enter a wound area by day 4 or 5 and become important modulators of the healing process. An intact T-cell immune system is essential, at least indirectly, for a normal healing outcome. Other cells, like mast cells, and their major protease, chymase, also play a role in the wound healing process by promoting capillary outgrowth and collagen formation. Again, these processes react well on treatment with NFηB down-regulating peptides. It has also been suggested that dermal dendritic cells participate in wound repair by initiating the inflammatory response and by stimulating epithelial proliferation and restoration of epithelial architecture. However, part of their function is now taken over by providing the healing wound with regular treatments with an NFκB down regulating peptide, supplemented by treatment with an angiogenesis modulating peptide.

The crucial pathophysiologic event that precipitates systemic inflammation is tissue damage. This can occur both as a result of the direct injury to tissues from mechanical or thermal trauma as well as cellular injury induced by mediators of ischemia-reperfusion injury such as oxygen free radicals. Injury results in the acute release of proinflammatory cytokines. If injury is severe, such as in extensive thermal injury, a profound release of cytokines occurs, resulting in the induction of a systemic inflammatory reaction, of which disseminated intravascular coagulation is often seen at on or more stages of the healing process. The ability of the host to adapt to this systemic inflammatory response is dependent on the magnitude of the response, the duration of the response, and the adaptive capacity of the host. Factors that have been implicated in prolongation of SIRS include under resuscitation in the acute phase following thermal injury, persistent or intermittent infection, ongoing tissue necrosis, and translocation of endotoxin across the bowel.

In one embodiment, the invention is providing a method and means to treat the systemic reaction to burns injuries by providing a subject believed to be in need thereof with a pharmaceutical composition comprising a NF-κB down-regulating peptide or functional analogue thereof and an agent directed against disseminated intravascular coagulation. Such an agent may for example be a composition comprising heparin, however, in a preferred embodiment, the invention provides treatment with a hypotonic pharmaceutical composition comprising a NF-κB down-regulating peptide or functional analogue thereof. Such treatment may for example comprise infusions with Ringer's lactate for the first 24 hours, the Ringer's lactate provided with, preferably, 1-1000 mg/l NFκB regulating peptide such as VLPALPQVVC (SEQ ID NO: 21), LQGVLPALPQ (SEQ ID NO: 22), LQG, LQGV (SEQ ID NO: 1), or VLPALP (SEQ ID NO: 6), or mixtures of two or more of such peptides. At this stage, it is important to keep the volume up, and, if needed, provide the peptide or functional analogue thereof in even further hypotonic solutions, such as 0.3 to 0.6% saline. NFκB regulating peptide can be given in the same infusion, the peptide (or analogue) concentration preferably being from about 1 to about 1000 mg/l, but the peptide can also been given in a bolus injection. Doses of 1 to 5 mg/kg body weight, for example every eight hours in a bolus injection or per infusionem until the patient stabilizes, are recommended. For example in cases where large affected areas are expected or diagnosed, it is preferred to monitor cytokine profiles, such as TNF-α or IL-10 levels, arachidonic acid metabolites an NO in the plasma of the treated patient, and to stop treatment when these levels are considered within normal boundaries. In another embodiment, it is herein provided to modulate a burn injury in a subject comprising providing the subject with a signaling molecule comprising a gene-regulatory peptide or functional analogue thereof wherein the subject is also provided with an agent directed against disseminated intravascular coagulation, in particular wherein the agent comprises Activated Protein C activity. Such an agent to modulate disseminated intravascular coagulation (DIC) comprises preferably (recombinant) human Activated Protein C. It is preferably given to the patient per infusionem, whereby NFκB regulating peptide can be given in the same infusion, the peptide (or analogue) concentration preferably being from about 1 to about 1000 mg/l, but the peptide can also been given in a bolus injection. Doses of 1 to 5 mg/kg body weight, for example every eight hours in a bolus injection or per infusionem until the patient stabilizes, are recommended.

The invention provides a method for modulating a burn injury in a subject comprising providing the subject with a signaling molecule comprising a gene-regulatory peptide or functional analogue thereof, in particular wherein the signaling molecule down-regulates translocation and/or activity of a gene transcription factor, especially wherein the gene transcription factor comprises an NF-κB/Rel protein, particularly wherein translocation and/or activity of the NF-κB/Rel protein is inhibited. Such peptides may be selected from peptides having NFκB down- or up-regulating activity in LPS stimulated RAW264.7 cells. More gene-regulating peptides and functional analogues can be found in a (bio)assay, such as a NFκB translocation assay as provided herein, and a by testing peptides for NFκB down- or up-regulating activity in LPS-stimulated or unstimulated RAW264.7 cells. For anti-inflammatory treatment, it is preferred that the peptide is selected from the group of peptides having NFκB down-regulating activity in LPS stimulated RAW264.7 cells, especially when the subject is at risk to experience a systemic inflammatory response syndrome occurring after the burn injury. Furthermore, a method is provided wherein the subject is also provided with an agent directed against disseminated intravascular coagulation, such as wherein the agent comprises Activated Protein C activity.

In response to a variety of pathophysiological and developmental signals, the NFκB/Rel family of transcription factors are activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFκB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or a modification or derivative thereof capable of modulating the activation of members of the NFκB/Rel family of transcription factors. Activation of NFκB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of genes that are under the control of NFκB/Rel family of transcription factors. Modulating comprises the upregulation or the downregulation of transcription. In a preferred embodiment, a peptide according to the invention, or a functional derivative or analogue thereof is used for the production of a pharmaceutical composition. Such peptides are preferably selected from group of peptides having NFκB down-regulating activity in LPS stimulated RAW264.7 cells. Examples of useful NFκB down-regulating peptides to be included in such a pharmaceutical composition are VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VVC, MTR and circular LQGVLPALPQVVC (SEQ ID NO: 17). More gene-regulating peptides and functional analogues can be found in a (bio)assay, such as a NFκB translocation assay as provided herein, which can also be used to further identify peptides having NFκB up-regulating activity in LPS stimulated RAW264.7 cells. Most prominent among NFκB down-regulating peptides are VLPALPQVVC (SEQ ID NO: 21), LQGVLPALPQ (SEQ ID NO: 22), LQG, LQGV (SEQ ID NO: 1), and VLPALP (SEQ ID NO: 6). These are also capable of reducing production of NO by a cell. It is herein also provided to use a composition that comprises at least two oligopeptides or functional analogues thereof, each capable of down-regulating NFκB, and thereby reducing production of NO and/or TNF-α by a cell, in particular wherein the at least two oligopeptides are selected from the group LQGV (SEQ ID NO: 1), AQGV (SEQ ID NO: 2) and VLPALP (SEQ ID NO: 4). Useful NFκB up-regulating peptides are VLPALPQ (SEQ ID NO: 13), GVLPALP (SEQ ID NO: 16) and MTRV (SEQ ID NO: 20). As indicated, more gene-regulatory peptides may be found with an appropriate (bio)assay. A gene-regulatory peptide as used herein is preferably short. Preferably, such a peptide is 3 to 15 amino acids long, more preferably, wherein the lead peptide is 3 to 9 amino acids long, most preferred wherein the lead peptide is 4 to 6 amino acids long, and capable of modulating the expression of a gene, such as a cytokine, in a cell. In a preferred embodiment, a peptide is a signaling molecule that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-κB in an NF-κB assay, or AP-1 in an AP-1 assay, or by another method as provided herein. Fragments can be somewhat (i.e. 1 or 2 amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a signaling molecule capable of modulating gene expression. Of course, such replacement scanning or mapping can be undertaken with amino acids other than Ala as well, for example with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptide. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty. The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect. In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can for example be performed in the process of gene-regulatory peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV (SEQ ID NO: 1), AQGV (SEQ ID NO: 2), LQGA (SEQ ID NO: 3), VLPALP (SEQ ID NO: 4), ALPALP (SEQ ID NO: 5), VAPALP (SEQ ID NO: 6), ALPALPQ (SEQ ID NO: 7), VLPAAPQ (SEQ ID NO: 8), VLPALAQ (SEQ ID NO: 9), LAGV (SEQ ID NO: 10), VLAALP (SEQ ID NO: 11), VLPALA (SEQ ID NO: 12), VLPALPQ (SEQ ID NO: 13), VLAALPQ (SEQ ID NO: 14), VLPALPA (SEQ ID NO: 15), GVLPALP (SEQ ID NO: 16), VVCNYRDVRFESIRLPGCPRGVNPVVSYAVALSCQCAL (SEQ ID NO: 24), RPRCRPINATLAVEKEGCPVCITVNTTICAGYCPT (SEQ ID NO: 25), SKAPPPSLPSPSRLPGPS (SEQ ID NO: 26), LQGVLPALPQVVC (SEQ ID NO: 17), SIRLPGCPRGVNPVVS (SEQ ID NO: 27), LPGCPRGVNPVVS (SEQ ID NO: 18), LPGC (SEQ ID NO: 19), MTRV (SEQ ID NO: 20), MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the α-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/min; absorbance was detected from 190-370 mm. There were different gradient systems used. For example for peptides LQG and LQGV (SEQ ID NO: 1): 10 minutes 100% A followed by linear gradient 0-10% B in 50 minutes. For example for peptides VLPALP (SEQ ID NO: 4) and VLPALPQ (SEQ ID NO: 7): 5 minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% C02 using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After 8 h of cultures cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 s, left standing at 4° C. for 15 min, and centrifuged at 15,000 rpm for 2 min. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 min on ice, then the lysates were centrifuged at 15,000 rpm for 2 min. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′) (SEQ ID NO: 28) synthesized to represent the NF-κB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 min at room temperature. The DNA-protein complex was resolved from free oligonucleotide by electrophoresis in a 6% polyacrylamide gel. The gels were dried and exposed to x-ray films.

The transcription factor NF-κB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-κB into the nucleus, on these extracts EMSA was performed. Here we see that indeed some peptides are able to modulate the translocation of NF-κB since the amount of labeled oligonucleotide for NF-κB is reduced. In this experiment peptides that show the modulation of translocation of NF-κB are: VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VLPALPQ (SEQ ID NO: 13), GVLPALP (SEQ ID NO: 16), VVC, MTRV (SEQ ID NO: 20), MTR.

RAW 264.7 mouse macrophages were cultured in DMEM, containing 10% or 2% FBS, penicillin, streptomycin and glutamine, at 37° C., 5% CO₂. Cells were seeded in a 12-wells plate (3×1106 cells/ml) in a total volume of 1 ml for 2 hours and then stimulated with LPS (E. coli 026:B6; Difco Laboratories, Detroit, Mich., USA) and/or NMPF (1 microgr/ml). After 30 minutes of incubation plates were centrifuged and cells were collected for nuclear extracts. Nuclear extracts and EMSA were prepared according to Schreiber et al. Cells were collected in a tube and centrifuged for 5 minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 was added and the sample was centrifuged (2 minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For electrophoretic mobility shift assays an oligonucleotide representing NF-κB binding sequence (5′-AGC TCA GAG GGG GAC TTT CCG AGA G-3′) (SEQ ID NO: 28) was synthesized. Hundred pico mol sense and antisense oligo were annealed and labeled with γ-³²P-dATP using T4 polynucleotide kinase according to manufacture's instructions (Promega, Madison, Wis.). Nuclear extract (5-7.5 μg) was incubated for 30 minutes with 75000 cpm probe in binding reaction mixture (20 microliter) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, 2-4 hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptides are able to modulate the basal as well as LPS induced levels of NF-κB. In this experiment peptides that show the inhibition of LPS induced translocation of NF-κB are: VLPALPQVVC (SEQ ID NO: 21), LQGVLPALPQ (SEQ ID NO: 22), LQG, LQGV (SEQ ID NO: 1), GVLPALPQ (SEQ ID NO: 23), VLPALP (SEQ ID NO: 6), VVC, MTR and circular LQGVLPALPQVVC (SEQ ID NO: 17). Peptides that in this experiment promote LPS induced translocation of NF-κB are: VLPALPQ (SEQ ID NO: 9), GVLPALP (SEQ ID NO: 16) and MTRV (SEQ ID NO: 20). Basal levels of NF-κB in the nucleus was decreased by VLPALPQVVC (SEQ ID NO: 21), LQGVLPALPQ (SEQ ID NO: 22), LQG and LQGV (SEQ ID NO: 1) while basal levels of NF-κB in the nucleus was increased by GVLPALPQ (SEQ ID NO: 23), VLPALPQ (SEQ ID NO: 9), GVLPALP (SEQ ID NO: 16), VVC, MTRV (SEQ ID NO: 20), MTR and LQGVLPALPQVVC (SEQ ID NO: 17). In other experiments, QVVC also showed the modulation of translocation of NF-κB into nucleus (data not shown).

Further modes of identification of gene-regulatory peptides by NFκB analysis.

Cells: Cells will be cultured in appropriate culture medium at 37° C., 5% CO₂. Cells will be seeded in a 12-wells plate (usually 1×10⁶ cells/ml) in a total volume of 1 ml for 2 hours and then stimulated with regulatory peptide in the presence or absence of additional stimuli such as LPS. After 30 minutes of incubation plates will be centrifuged and cells are collected for cytosolic or nuclear extracts.

Nuclear Extracts: Nuclear extracts and EMSA could be prepared according to Schreiber et al. Method (Schriber et al. 1989, Nucleic Acids Research 17). Cells are collected in a tube and centrifuged for 5 minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 is added and the sample is centrifuged (2 minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′) (SEQ ID NO: 28) are synthesized. Hundred pico mol sense and antisense oligo are annealed and labeled with γ-³²P-dATP using T4 polynucleotide kinase according to manufacture's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 μl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex is resolved from free oligonucleotide by electrophoresis in a 4-6% polyacrylamide gel (150 V, 2-4 hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 miroliter Neutr-Avidin-plus beads for 1 h at 4° C. with constant shaking. Beads are washed five times with lysis buffer by centrifugation at 6000 rpm for 1 min. Proteins are eluted by incubating the beads in 0.05 N NaoH for 1 min at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-κB subunit are pooled and concentrated. Fractions are then dissolved in appropriate volume and could be analyzed on mass-spectrometry.

Further references: PCT International Patent Publications WO99/59671, WO01/72831, WO97/49721, WO01/10907, and WO01/11048, the content of the entirety of all of which are incorporated by this reference.

What is claimed is:
claims 11 and 15 have been amended herein. All of the pending claims 1 through 20 are presented below. This listing of claims will replace all prior versions and listings of claims in the application. Please enter these claims as amended.

1. (original) A method for modulating a burn injury in a subject, said method comprising:
providing the subject with a gene-regulatory peptide or functional analogue thereof.

2. (original) The method according to claim 1 wherein said gene-regulatory peptide or functional analogue thereof down-regulates translocation, activity, or translocation and activity of a gene transcription factor.

3. (original) The method according to claim 2 wherein said gene transcription factor comprises an NF-kappaB/Rel protein.

4. (original) The method according to claim 2 wherein translocation, activity, or translocation and activity of NF-kappaB/Rel protein is inhibited.

5. (original) The method according to claim 3 wherein translocation, activity, or translocation and activity of NF-kappaB/Rel protein is inhibited.

6. (original) The method according to claim 1 wherein said gene-regulatory peptide or functional analogue thereof has NFkappaB down-regulating activity in LPS stimulated RAW264.7 cells.

7. (original) The method according to claim 2 wherein said gene-regulatory peptide or functional analogue thereof has NFkappaB down-regulating activity in LPS stimulated RAW264.7 cells.

8. (original) The method according to claim 3 wherein said gene-regulatory peptide or functional analogue thereof has NFkappaB down-regulating activity in LPS stimulated RAW264.7 cells.

9. (original) The method according to claim 4 wherein said gene-regulatory peptide or functional analogue thereof has NFkappaB down-regulating activity in LPS stimulated RAW264.7 cells.

10. (original) The method according to claim 5 wherein said gene-regulatory peptide or functional analogue thereof has NFkappaB down-regulating activity in LPS stimulated RAW264.7 cells.

11. (amended) The method according to any one of claims 1 to 10 claim 1 wherein the subject is at risk of suffering a systemic inflammatory response syndrome occurring after the burn injury.

12. (original) The method according to claim 11 wherein said gene-regulatory peptide or functional analogue thereof has NFkappaB down-regulating activity in LPS unstimulated RAW264.7 cells.

13. (original) The method according to claim 1 further comprising:
providing the subject with an agent directed against disseminated intravascular coagulation.

14. (original) The method according to claim 13 wherein said agent has Activated Protein C activity.

15. (amended) The method according to any one of claims 2 through 12 claim 2 further comprising:
providing the subject with an agent directed against disseminated intravascular coagulation.

16. (original) The method according to claim 15 wherein said agent has Activated Protein C activity.

17. (original) A pharmaceutical composition comprising:
a NF-kappaB down-regulating peptide or functional analogue thereof, and
an agent directed against disseminated intravascular coagulation.

18. (original) A hypotonic pharmaceutical composition comprising:
a NF-kappaB down-regulating peptide or functional analogue thereof.

19. (original) A pharmaceutical composition comprising:
a NF-kappaB down-regulating peptide or functional analogue thereof, and
a bacteriostatic compound comprising silver.

20. (original) A method for treating a subject suffering from a burn, the method comprising:
providing the subject with a sufficient amount of a gene-regulatory peptide to down-regulate translocation, activity, or translocation and activity of NF-kappaB/Rel protein, and
further providing the subject with an agent directed against disseminated intravascular coagulation, said agent having Activated Protein C activity.


SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC